(19) United States
(12) Patent Application Publication
(10) Pub. No.: US 2023/0356005 A1
(43) Pub. Date: Nov. 9, 2023

(54) Localization of Biomineralizations Using Diminished-Frequency Spectral Signatures

(71) Applicant: Applaud Medical, Inc., San Francisco, CA (US)

(72) Inventors: William Behnke-Parks, San Francisco, CA (US); Yuri Pishchalnikov, Rohnert Park, CA (US); Ishan Ramaiah, Sunnyvale, CA (US); Paidamoyo Ewing, Burlingame, CA (US); Matthew Mellema, Davis, CA (US)

(21) Appl. No.: 18/312,119

(22) Filed: May 4, 2023

Related U.S. Application Data
(60) Provisional application No. 63/364,133, filed on May 4, 2022.

(51) Int. Cl. A61N 7/00

(52) U.S. Cl. CPC A61N 7/00; A61N 2007/0039; A61N 2007/0073

(57) ABSTRACT

A method for localization a biomineralization in a volume comprises (a) producing, by an ultrasonic transducer, pulses of produced ultrasonic energy waves having a fundamental frequency; (b) injecting microbubbles proximal to the biomineralization; (c) receiving, by an acoustic receiver, returned ultrasonic energy waves to produce a signal output; (d) processing the signal output to isolate diminished frequencies of the signal output, the diminished frequencies having a frequency range that is less than 50% of the fundamental frequency and greater than or equal to about 4% of the fundamental frequency; (e) monitoring the diminished frequencies, with a processor, for a diminished-frequency spectral signature that corresponds with a location of the biomineralization; and (f) determining a spatial location of the biomineralization, with the processor, based on the diminished-frequency spectral signature.

### CROSS-REFERENCE TO RELATED APPLICATIONS

This application claims priority to U.S. Provisional Application No. 63/364,133, titled “Localization of Biomineralizations Using Subharmonic Spectral Signatures,” filed on May 4, 2022, which is hereby incorporated by reference.

### TECHNICAL FIELD

This invention relates generally to the treatment of undesired biomineralizations using microbubbles which are excited or cavitated using ultrasound energy.

### BACKGROUND

A large number of pathologies are characterized in part by the presence of pathological biomineralizations. Examples include urinary stones, biliary stones, blood clots, fibroids, bone spurs, and atheromatous plaques.

Ultrasound can be used to localize biomineralizations in situ. It would be desirable to improve the ability to localize biomineralizations in situ.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages and novel features of the disclosure will be set forth in the following detailed description of the disclosure when considered in conjunction with the drawings, which are intended to illustrate, not limit, the invention.

An aspect of the invention is directed to a method for localizing a biomineralization in a volume. The method includes producing, by an ultrasonic transducer, pulses of produced ultrasonic energy waves having a fundamental frequency; injecting an ensemble of microbubbles proximal to the biomineralization; receiving, by an acoustic receiver, returned ultrasonic energy waves to detect a broadband signal output; processing the broadband signal output to isolate diminished frequencies of the broadband signal output, the diminished frequencies lower than 50% of the fundamental frequency; monitoring the diminished frequencies, with a processor, for a diminished-frequency spectral signature that corresponds with a location of the biomineralization; and determining a spatial location of the biomineralization, with the processor, based on the diminished-frequency spectral signature.

In one or more embodiments, the diminished frequencies are less than or equal to 25% of the fundamental frequency. In one or more embodiments, the fundamental frequency is greater than or equal to about 250 kHz and less than about 1 MHz. In one or more embodiments, the fundamental frequency is less than or equal to about 750 kHz. In one or more embodiments, the fundamental frequency is about 500 kHz. In one or more embodiments, the diminished frequencies are greater than or equal to about 20 kHz and less than or equal to about 120 kHz.

In one or more embodiments, the diminished-frequency spectral signature includes an increased acoustic pressure response of the diminished frequencies when the produced ultrasonic energy waves are aligned with the biomineralization compared to when the produced ultrasonic energy waves are offset from the biomineralization. In one or more embodiments, the increased acoustic pressure response of the diminished frequencies occurs when the microbubbles accumulate on a surface of the biomineralization. In one or more embodiments, the increased acoustic pressure response of the diminished frequencies occurs when the microbubbles form a microbubble cloud and/or a microbubble cluster.

In one or more embodiments, the diminished-frequency spectral signature includes an integrated signal of an acoustic pressure of the diminished frequencies. In one or more embodiments, the method further comprises moving the ultrasound transducer axially with respect to the volume while the ultrasound transducer produces the pulses of produced ultrasonic energy waves, wherein the diminished-frequency spectral signature includes a first increase in a diminished-frequency response over a first time period, the first increase in the diminished-frequency response compared to a background response signal, a decrease in the diminished-frequency response over a second time period compared to the diminished-frequency response over the first time period, and a second increase in the diminished-frequency response over a third time period, the second increase in the diminished frequency response compared to the background response signal. The second time period immediately follows the first time period. The third time period immediately follows the second time period.

In one or more embodiments, the decrease in the diminished-frequency response over the second time period corresponds to the spatial location of the biomineralization. In one or more embodiments, the first and second increases in the diminished-frequency response correspond to a spatial location of microbubbles and/or a microbubble cloud.

In one or more embodiments, the diminished frequencies are greater than or equal to about 4% of the fundamental frequency. In one or more embodiments, the method further comprises amplifying the diminished frequencies of the broadband signal output.

In one or more embodiments, the processing step includes filtering, in a low-pass filter or a bandpass filter, the broadband signal output to isolate the diminished frequencies. In one or more embodiments, the processing step includes performing, with the processor, a fast Fourier transform of the broadband signal output with respect to the diminished frequencies.

Another aspect of the invention is directed to a system for localizing a biomineralization in a volume. The system comprises an ultrasound device that produces pulses of produced ultrasonic energy waves having a fundamental frequency; a receiver that receives returned ultrasonic energy waves and produces signals that represent the returned ultrasonic energy waves; a low-pass filter configured to receive the signal output of the receiver and to isolate diminished frequencies of the signal output, the diminished frequencies less than 50% of the fundamental frequency; an analog-to-digital converter configured to convert the diminished frequencies to digital diminished-frequency representations; a non-volatile computer-readable memory that stores the digital diminished-frequency representations; a catheter configured to inject an ensemble of microbubbles proximal to the biomineralization; and a computer having a microprocessor and non-volatile computer-readable memory, the computer operatively coupled to the computer-readable memory, the non-volatile computer-readable memory storing computer-readable instructions that, when executed by the microprocessor, cause the microprocessor to automatically determine whether the digital diminished-frequency representations include a diminished-frequency spectral signature that corresponds with a spatial location of a biomineralization in a volume, and produce an output control signal that indicates that the diminished-frequency spectral signature is detected.

In one or more embodiments, the system further comprises a preamplifier having an input electrically coupled to an output of the receiver, the preamplifier having an output electrically coupled to an input of the low-pass filter; and a gain amplifier having an input electrically coupled to an output of the low-pass filter, the gain amplifier having an output electrically coupled to an input of the analog-to-digital converter.

In one or more embodiments, the system further comprises a field-programmable gate array (FPGA) in electrical communication with the memory, the FPGA configured to produce a trigger signal to store the digital diminished-frequency representations in the memory.

In one or more embodiments, the system further comprises the analog-to-digital converter is a first analog-to-digital converter, the non-volatile computer-readable memory is a first non-volatile computer-readable memory, and the system further comprises a high-pass filter configured to receive the signal output of the receiver and to isolate the fundamental and harmonic frequencies of the signal output; a second analog-to-digital converter configured to convert the fundamental and harmonic frequencies to digital fundamental and harmonic representations; and a second non-volatile computer-readable memory that stores the digital fundamental and harmonic representations. The computer-readable instructions, when executed by the microprocessor, further cause the microprocessor to produce a diagnostic image on a display screen in electrical communication with the computer, the diagnostic image corresponding to the digital fundamental and harmonic representations.

In one or more embodiments, the ultrasound device and the receiver are coaxially aligned such that the produced ultrasonic energy waves from the ultrasound device pass through the receiver before passing into the volume. In one or more embodiments, the diminished frequencies are greater than or equal to about 4% of the fundamental frequency.

Another aspect of the invention is directed to a system for localizing a biomineralization in a volume. The system comprises an ultrasound device that produces pulses of produced ultrasonic energy waves having a fundamental frequency; a receiver that receives returned ultrasonic energy waves and produces signals that represent the returned ultrasonic energy waves; an analog-to-digital converter configured to convert the signals to digital frequency representations; a non-volatile computer-readable memory that stores the digital frequency representations; a catheter configured to inject an ensemble of microbubbles proximal to the biomineralization; and a computer having a microprocessor and non-volatile computer-readable memory, the computer operatively coupled to the segmented computer-readable memory, the non-volatile computer-readable memory storing computer-readable instructions that, when executed by the microprocessor, cause the microprocessor to automatically perform a fast Fourier transform of the digital frequency representations with respect to diminished frequencies that are less than 50% of the fundamental frequency to isolate the diminished frequencies, determine whether a diminished-frequency signal corresponding to the diminished frequencies includes a diminished-frequency spectral signature that corresponds with a spatial location of a biomineralization in a volume, and produce an output control signal that indicates that the diminished-frequency spectral signature is detected.

### BRIEF DESCRIPTION OF THE DRAWINGS

Fora fuller understanding of the nature and advantages of the concepts disclosed herein, reference is made to the detailed description of preferred embodiments and the accompanying drawings.

FIG. 1 is a simplified diagram of an ultrasound device for locating a biomineralization in a target region.

FIGS. 2A and 2B illustrate an example representation of a method for monitoring acoustic emissions at the diminished frequency range where a diminished-frequency spectral signal signature can be used to detect the location of the urinary stone and/or to position the catheter near the urinary stone.

FIG. 3 is a plot of power spectral density versus frequency with and without microbubbles.

FIG. 4 is a plot of power spectral density versus time with and without an microbubbles.

FIGS. 5A and 5B are images of microbubbles clustering into clouds.

FIG. 6 is a plot of spectral magnitude versus frequency in different conditions.

FIG. 7 is a plot of the fundamental frequency component on the broadband sensor versus time in different conditions.

FIG. 8 is a plot of the integrated power signal versus time for the 80 kHz diminished-frequency signal in different conditions.

FIG. 9 is a block diagram of a system for detecting diminished-frequency spectral signatures according to an embodiment.

FIG. 10 is a block diagram of a system for simultaneously detecting multiple frequency ranges according to an embodiment.

FIG. 11 is a flow chart of a method for performing ultrasound therapy on a patient that has an undesired biomineralization, such as a urinary stone, according to an embodiment.

FIGS. 12 and 13 are integrated plots of power spectral density integrated over two example diminished-frequency bandwidths versus time at different initial number densities and with and without an artificial kidney stone present in a test vessel.

### DETAILED DESCRIPTION

Microbubbles are injected into a target region where an undesired biomineralization is located. The target region is sonicated with low-megahertz or sub-megahertz frequency (e.g., having a center or fundamental frequency less than about 1 megahertz (MHz)), and the acoustic response is processed and monitored in the diminished frequency range (e.g., less than 50% of the fundamental frequency). A unique diminished-frequency spectral signature of the acoustic response indicates when the microbubbles are clustered on the biomineralization surface, such as in a microbubble ensemble and/or a microbubble cloud. The diminished-frequency spectral signature can be used for biomineralization localization prior to therapeutic treatment.

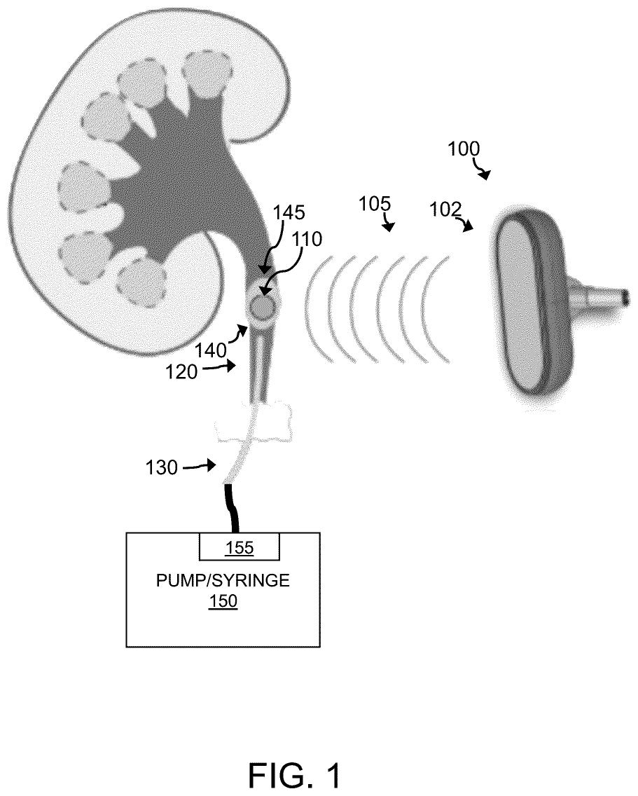

FIG. 1 is a simplified diagram of an ultrasound device or treatment head 100 comprising one or more ultrasound transducers 102 that produce ultrasound energy 105 that is directed towards a target region containing an unwanted biomineralization, such as a urinary stone 110 lodged in a patient's ureter 120. The ultrasound device 100 is controllable and can be configured and arranged to be activated or deactivated (ON or OFF) so as to temporally modulate the intensity of the ultrasound energy 105 at the target region. The ultrasound device 100 may be pulsed or operated with a desired duty cycle or otherwise programmed to produce desired sequences of ultrasound energy 105. The ultrasound device 100 is configured to produce ultrasound energy 105 having a center or fundamental frequency (f₀) that is less than about 1 megahertz (MHz) and greater than or equal to about 200 kilohertz (kHz). For example, the fundamental frequency can be greater than or equal to about 250 kHz and less than or equal to about 750 kHz. In a specific example, the fundamental frequency of the ultrasound energy 105 is about 500 kHz. As used herein, “about” means plus or minus 10% of the relevant value.

A catheter 130 can be inserted into the ureter 120 (or another volume or region of interest) to introduce an ensemble of microbubbles 140, such as engineered microbubbles, that can cluster on and/or around the urinary stone 110 (e.g., as stone-surface-accumulating (SSA) microbubbles) and can form a microbubble cloud 145. A pump or a syringe 150 can be fluidly coupled to the catheter 130 to inject the microbubbles 140 through the catheter 130. The pump or syringe 150 can include a reservoir 155 that contains the microbubbles. Alternatively, a microbubble reservoir can be located in the catheter 130.

The ultrasound energy 105 produced by the ultrasound device 100 causes the microbubbles 140 to repeatedly (e.g., cyclically) expand and inertially collapse. Each collapse can focus the insonation pressure to facilitate fragmentation and/or mechanical erosion of the urinary stone 110. Each collapse can also produce distinct acoustic emissions that can be detected through an ultrasound receiver or sensor that can be included in or distinct from the ultrasound device 100.

The ultrasound device 100 is configured to detect the acoustic emissions of (e.g., returned ultrasonic energy waves from) the collapsing microbubbles at a diminished frequency and/or at a diminished-frequency range. For example, when the center or fundamental frequency (i.e., f₀) of the ultrasound energy is 500 kHz, the diminished-frequency range can be about 20 kHz to about 240 kHz, including about 40 kHz, about 80 kHz, about 120 kHz, about 160 kHz, about 200 kHz, and any value or range between any two of the foregoing frequencies. In a specific example, the diminished-frequency range can be about 60 kHz to about 100 kHz, about 60 kHz to about 120 kHz, about 100 kHz to about 240 kHz, or another diminished-frequency range. In general, the diminished-frequency range is less than half of the fundamental frequency

$$( i . e . , \frac{{f}_{0}}{2} )$$

and can be greater than or equal to about 4% of f₀ or greater than or equal to about 5% of f₀, including about 10% of f₀, about 20% of f₀, about 30% of f₀ , about 40% of f₀, and/or any value or range between any two of the foregoing percentages of f₀. In a specific example, the diminished-frequency range can be greater than or equal to

$$\frac{{f}_{0}}{8}$$

and less than or equal to

$$\frac{{f}_{0}}{4}$$

and/or greater than or equal to

$$\frac{{f}_{0}}{4}$$

and less than

$$\frac{{f}_{0}}{2} .$$

A unique diminished-frequency acoustic emission signature observed in the diminished-frequency range can be used to monitor and/or localize treatment. For example, monitoring acoustic emissions (e.g., returned ultrasonic energy waves) at the diminished-frequency range can be used to monitor for proper placement of microbubbles during treatment, localization of the urinary stone 110, and/or for device configuration. Without being bound by theory, it is believed that the clustered microbubbles at the surface of the biomineralization form a microbubble cloud, which has a lower resonance frequency than individual microbubbles (or small microbubble clusters).

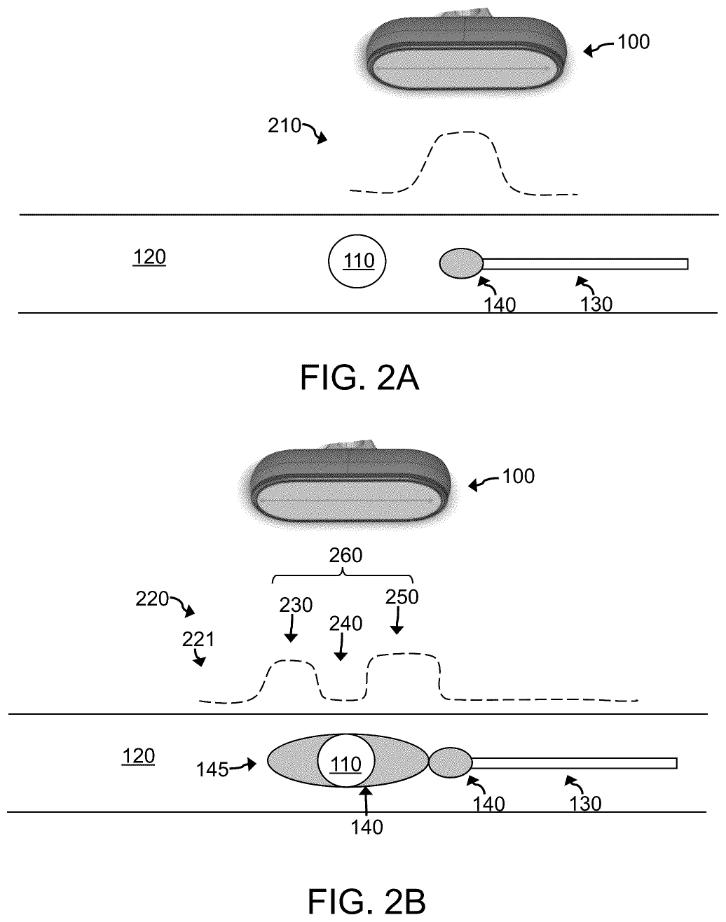

FIGS. 2A and 2B illustrate an example representation of a method for monitoring acoustic emissions at the diminished-frequency range where a diminished-frequency spectral signal signature can be used to localize the urinary stone 110 and/or to position the catheter 130 near the urinary stone 110. In FIG. 2A, the ultrasound device 100 is positioned in alignment with the catheter 130 as an ensemble microbubbles 140 is injected. The catheter 130 (and the ultrasound device 100) is positionally offset with respect to the urinary stone 110. A first diminished-frequency response 210 is observed.

In FIG. 2B, the ultrasound device 100 and the catheter 130 are repositioned. The ultrasound device 100 is positioned in alignment with the urinary stone 110. The catheter 130 is positioned sufficiently close to the urinary stone 110 such that an ensemble of microbubbles 140 accumulate on and/or around the surface of the urinary stone 110 to form a microbubble cloud 145. The ensemble can include various numbers of microbubbles ranging from several (e.g., a few) microbubbles to hundreds, thousands, millions, or billions (e.g., tens of billions) of microbubbles 140.

A second diminished-frequency response 220 is observed when the ultrasound device 100 is repositioned. The ultrasound device 100 can be physically moved to move the ultrasound beam and/or the ultrasound beam can be moved electronically. The second diminished-frequency response 220 includes a first increase or peak 230, a decrease or dip 240, and a second increase or peak 250. The first increase 230 can be determined with respect to or compared to a background response signal 221. The decrease 240 can be determined with respect to or compared to the first increase 230. The decrease 240 can be about equal to the background response signal 221. The second increase 250 can be determined with respect to or compared to the background response signal 221 and/or to the decrease 240. The second increase 250 is axially longer than the first increase 230, which corresponds to the microbubbles injected by the catheter 130 and the microbubble cloud 145 proximal to the catheter 130. The first increase 230 corresponds to the microbubble cloud 145 distal to the catheter 130. The decrease 240 corresponds to the urinary stone 110. The first increase 230, the decrease 240, and the second increase 250 in the second diminished-frequency response 220 can comprise a diminished-frequency spectral signature 260 of the target urinary stone 110 and associated microbubble cloud 145.

### First Example

To investigate SSA microbubble dynamics, the time-dependent acoustic emissions were recorded in a model vessel, with or without a model urinary stone, during insonation (P<1.4 MPa, f₀=0.5 MHz) at low-duty cycle (20 μs on-time, 100 Hz PRF (pulse repetition frequency)) in the presence or absence of SSA microbubbles. High-speed images were collected at 10,000 frames per second using a Shimadzu HPV-X2 camera mounted on a customized inverted Nikon microscope.

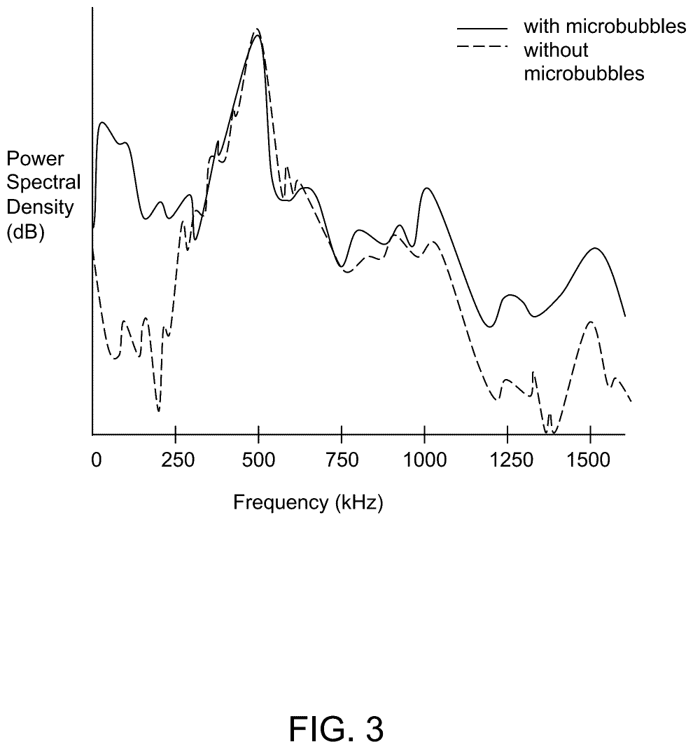

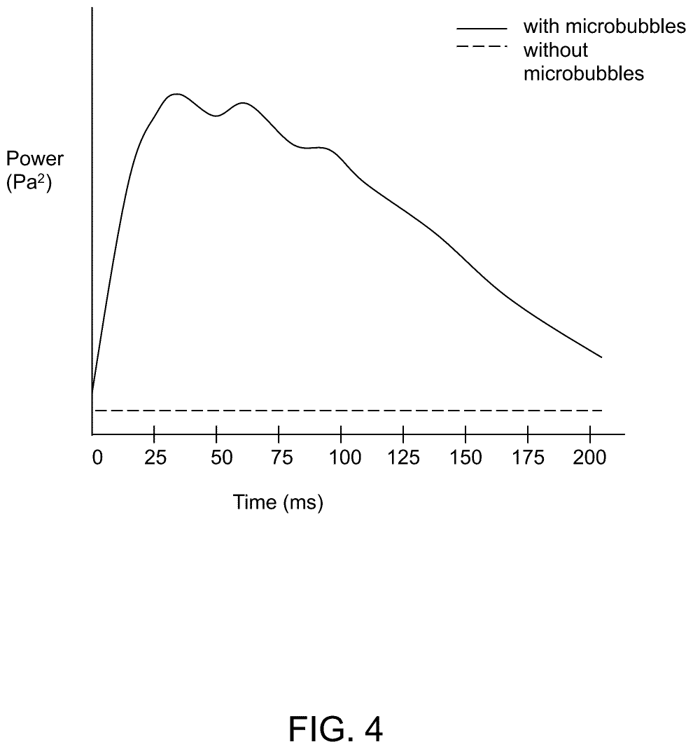

In addition to the expected acoustic emissions in the fundamental frequency domain (f₀) and in the harmonic range (2f₀, 3f₀), strong acoustic emissions were observed in the diminished-frequency range between 20 and 120 kHz with a high signal-to-background ratio (FIG. 3). While emissions in the harmonic range display a quadratic dependence on applied pressure, diminished-frequency emissions follow a stronger dependence on pressure, and a long lifetime (FIG. 4). FIG. 4 illustrates the power of the acoustic-emissions response in the bandwidth of 50 kHz to 250 kHz with and without microbubbles. Approximately 1 MPa of pressure is needed for an acoustic-emissions response in this bandwidth. It is believed that the acoustic-emissions response decreases over time as microbubbles are broken and/or pushed off the detection area (e.g., by acoustic wind). Additional microbubbles would need to be injected to maintain the acoustic-emissions response at about the same magnitude.

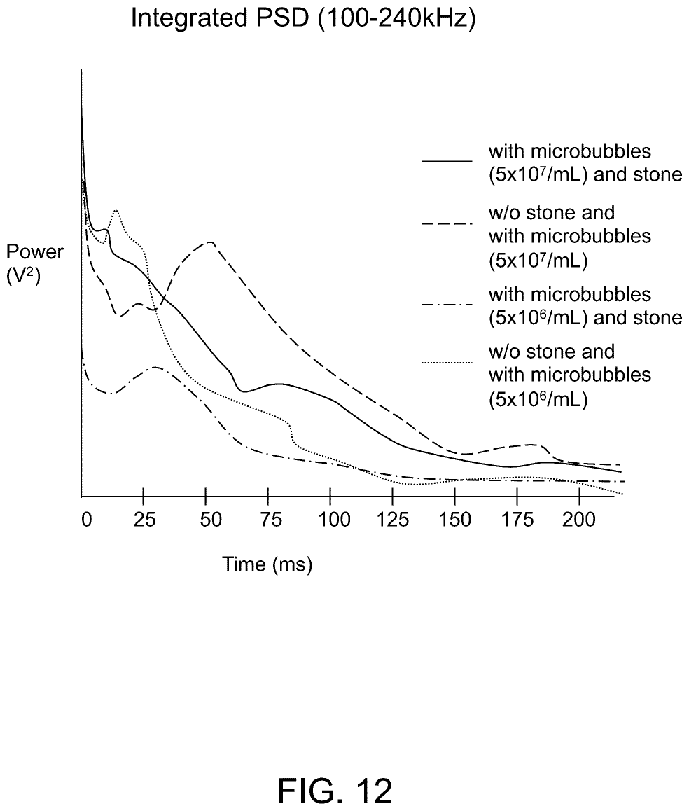

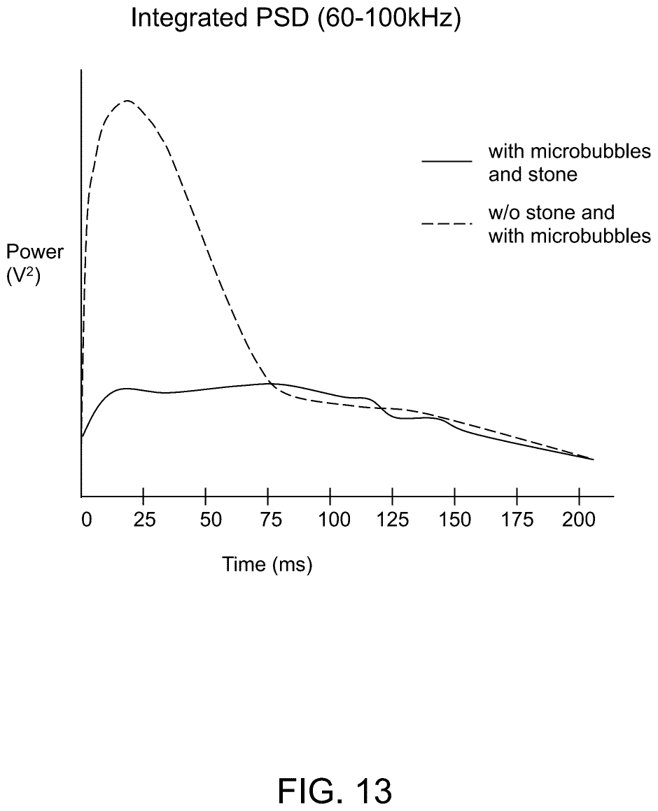

Furthermore, diminished-frequency emissions also displayed a dependence on initial number density before insonation, as illustrated in FIGS. 12 and 13. FIG. 12 is a plot of power spectral density (PSD) integrated over 100-240 kHz versus time with and without an artificial kidney stone in a test vessel with and without microbubbles when the fundamental frequency is 500 kHz. The initial number densities of microbubbles were 5×10⁶ microbubbles/mL and 5×10⁷ microbubbles/mL FIG. 13 is a plot of PSD integrated over 60-100 kHz versus time with and without an artificial kidney stone in a test vessel with and without microbubbles when the fundamental frequency is 500 kHz. The initial number densities of microbubbles were 5×10⁸ microbubbles/mL.

As can be seen in FIGS. 12 and 13, the diminished-frequency emissions are different in the presence of a stone versus off of a stone (i.e., no stone). For example, in FIG. 13 the no-stone plot has a sharp peak at about 25 ms followed by a sharp decline in amplitude from 25 ms to 75 ms at which point the no-stone and the stone plots are similar. In contrast, the stone plot in FIG. 13 has a small increase in amplitude between 0 and 25 ms after which the plot has a plateau in amplitude from about 25 ms to about 75 ms and then a slow decline in amplitude. In FIGS. 12 and 13 the stone plot and the no-stone plots are very different in profile, showing different temporal profiles and a strong initial number-density dependence. The stone plot can be considered as a low-noise “background,” which can allow for robust discrimination and detection of the stone. The initial number density diminished-frequency response can be a diminished-frequency spectral signature.

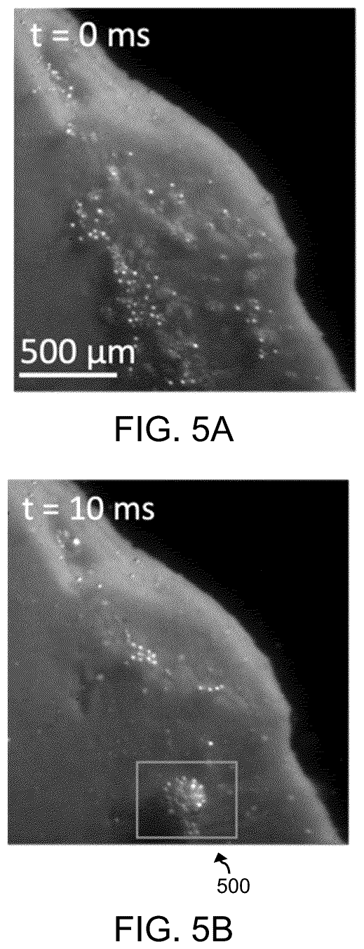

High-speed imaging observed clustering of SA microbubbles into clouds ˜100 μm in diameter on the millisecond timescale (FIGS. 5A, 5B). An example microbubbles cloud or cluster 500 is illustrated in FIG. 5B. The SSA microbubbles can be modeled as microbubble clouds to describe the origin of this diminished-frequency acoustic emission. The diminished-frequency range provided a distinct diminished-frequency signature with a strong signal-to-background ratio and a broadband signal in the diminished-frequency range (e.g., in the range below 250 kHz, i.e., less than 0.5f₀). This highly correlated microbubble-clustering effect is distinct from the uncorrelated and statistically-independent signals typical of diagnostic imaging approaches to microbubble-based detection.

### Second Example

To investigate SSA microbubble dynamics, synthetic urinary stones were insonated with diagnostic-like ultrasound pulse trains (P<1.4 MPa, f₀=0.5 MHz, PRF=1 kHz, N=50 pulses). Acoustic emissions were measured in the presence or absence of SSA microbubbles using passive cavitation detection (PCD) in situ. The PCD signals were analyzed for average magnitude of spectral components such as diminished-frequencies from 20 kHz to 250 kHz, fundamental from 470 kHz to 520 kHz, and broadband emissions from 1.6 to 1.9 MHz—a spectral signature previously correlated with inertial cavitation.

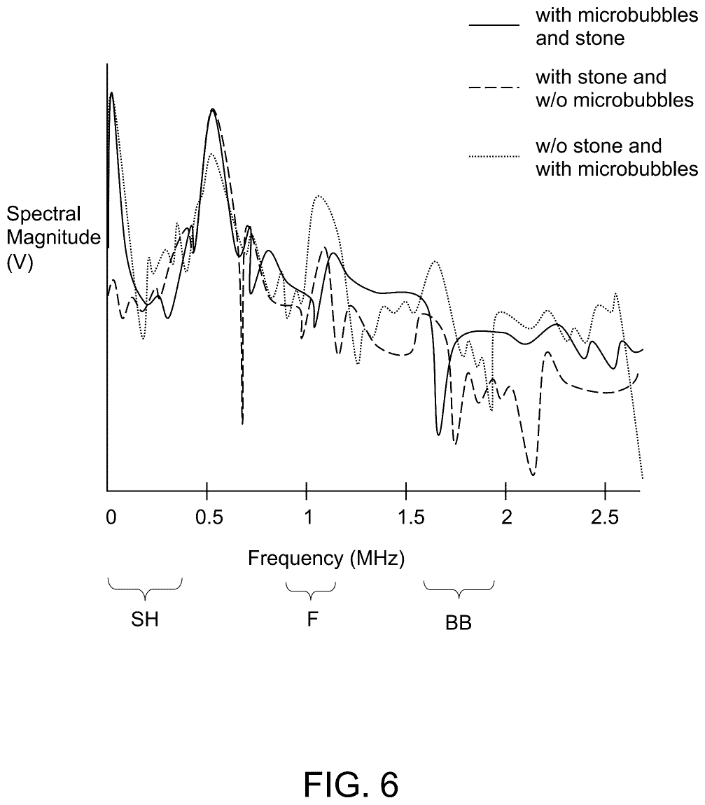

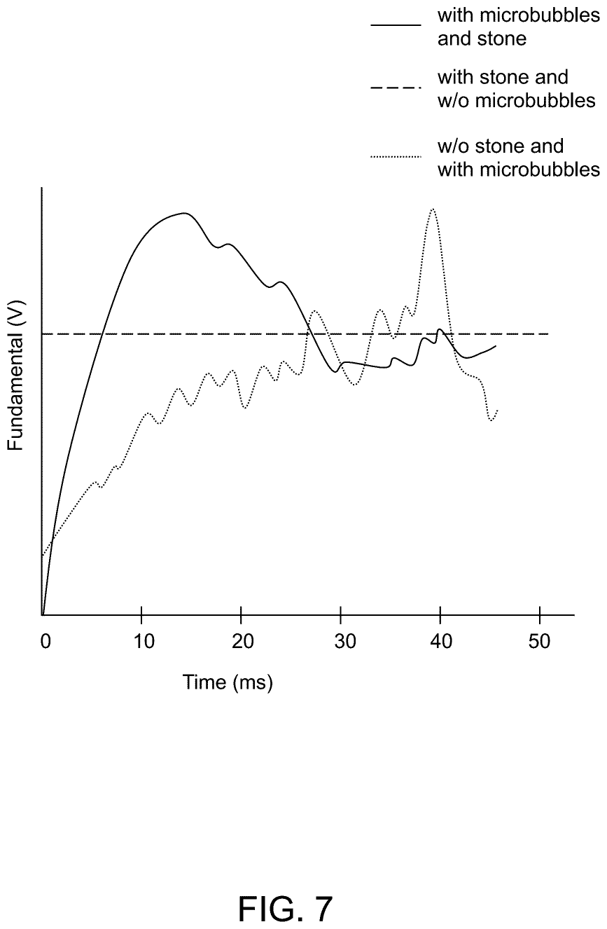

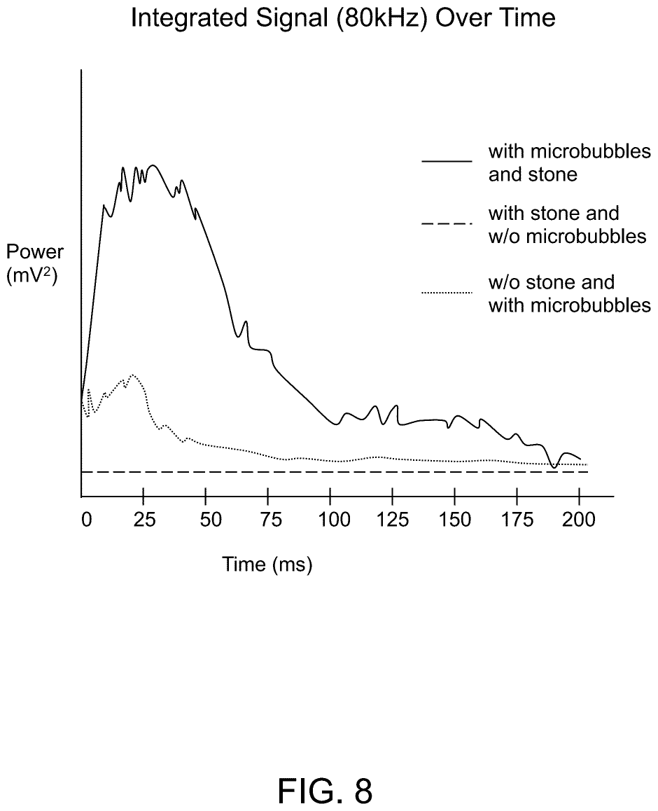

SSA microbubbles were observed to significantly alter diminished-frequency (SH or subharmonic), fundamental (F), and broadband (BB) emissions for the detection and monitoring of partially obstructing urinary stones (mean±SEM, N=5, as illustrated in FIG. 6). The diminished-frequency emissions are at frequencies that are less than half of the fundamental frequency. For example, the fundamental frequency is about 500 kHz and the diminished frequencies are about 50 kHz to about 250 kHz. The broadband-frequency emissions are at frequencies that are at harmonics of the fundamental frequency, such as 3 to 4 times the fundamental frequency. Relative to SSA microbubble free in solution (orange), the emissions from SSA microbubbles in proximity to partially occluding stones displayed a more rapid increase in the fundamental bandwidth (T=2.5±0.7 ms versus T=14.0±3 ms, as illustrated in FIG. 7), followed by a decay. The diminished-frequency range (FIG. 8) decayed more rapidly than the fundamental (FIG. 7) and the broadband emissions. Urinary stones in the absence of SSA microbubbles showed negligible time-dependence of spectral components (FIGS. 7-8). This approach of insonating with low-frequency ultrasound to produce strong SSA microbubble dynamics combined with spectral detection of emissions can be used for intra-treatment monitoring of microbubble-mediated therapy for urinary stone disease.

In general, the following observations of microbubble dynamics can be used (e.g., as diminished-frequency spectral signatures) to measure the presence or absence of a urinary stone: (1) there is an increased pressure at the urinary stone surface which drives the bubbles stronger and (2) there is a different time dependence on versus off the urinary stone. It may also be possible to introduce a higher local concentration of microbubbles on the urinary stone surface during placement of the catheter and/or of the ultrasound device, which can be followed by a wash step to remove the microbubbles. For example, saline or another liquid can be injected, through the catheter, into the ureter to wash away the microbubbles from the urinary stone surface, if desired. Additionally or alternatively, the spectral signal can be monitored to localize the urinary stone (e.g., as described above with respect to FIG. 2).

The diminished-frequency signal showed a higher amplitude and longer lifetime on a solid surface, i.e. a stone or biomineralization, versus off of the surface. Therefore the integrated signal over time has a strong signal-to-background ratio (e.g., as illustrated in FIG. 8). This integrated signal can be used (e.g., as a diminished-frequency spectral signature) to localize a stone or solid surface. Also, this strong signal is anti-correlated with returned ultrasound energy in the fundamental frequency range (the returned ultrasonic energy signals at f₀ is initially lower (e.g., as illustrated in FIG. 7) when returned ultrasonic energy signals in the diminished-frequency range are high). These signals may be used for passive detection of alignment and position by sweeping the beam and detecting a change in this acoustic signature and correlating this change with the insonation beam.

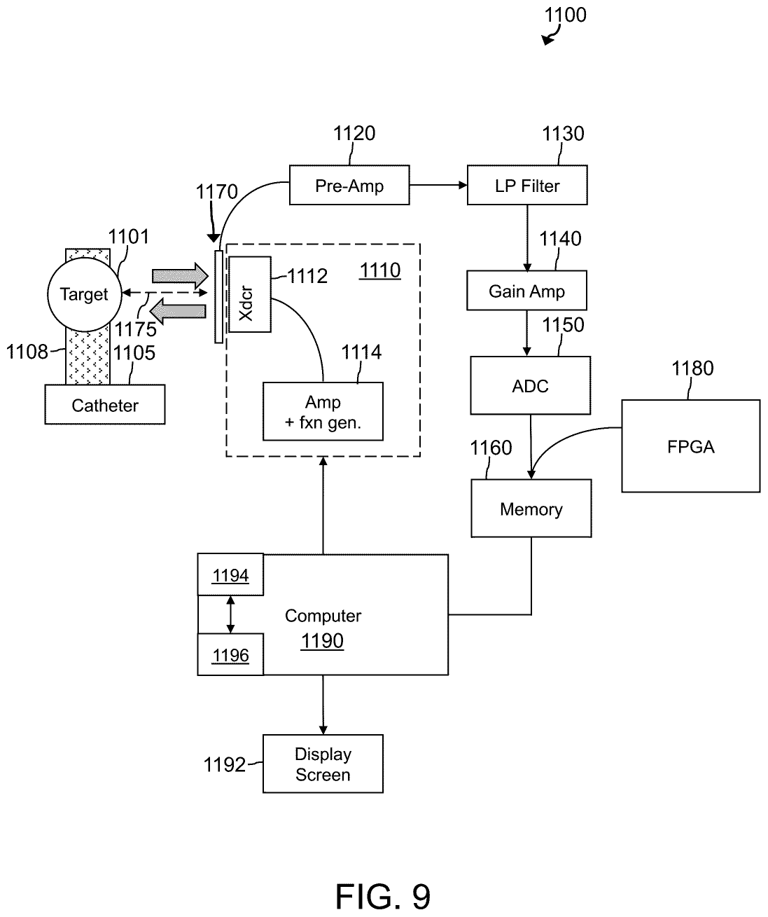

FIG. 9 is a block diagram of a system 1100 for localizing a biomineralization in a volume according to an embodiment. The system 1100 includes a catheter 1105, an ultrasound device 1110, a preamplifier 1120, a low-pass filter 1130, a gain amplifier 1140, an analog-to-digital controller (ADC) 1150, non-volatile memory 1160, and an acoustic receiver 1170. The ultrasound device 1110 includes one or more transducers 1112 that are electrically coupled to an amplifier and signal generator 1114. The amplifier and signal generator 1114 produces signals having a predetermined fundamental frequency at a predetermined PRF, a predetermined duty cycle, and a predetermined amplitude (e.g., acoustical pressure) for a predetermined duration. The ultrasound device 1110 can be the same as ultrasound device 100. The transducer(s) 1112 are driven by signals from the amplifier and signal generator 1114 to produce ultrasound energy.

The ultrasound device 1110 is preferably placed on the skin surface of the subject (e.g., via an acoustic coupling medium such as a gel) in the vicinity of a target 1101, such as a biomineralization. Additionally or alternatively, the ultrasound device 1110 can be placed on the surface that defines a volume or region of interest. In operation, the ultrasound device 1110 produces or emits acoustic energy having a fundamental frequency of less than about 1 MHz, such as about 250 kHz to about 750 kHz. In a specific example, the ultrasound energy can have a fundamental frequency of about 500 kHz. The ultrasound energy waves are returned from the target 1101, the microbubbles 1108 injected through the catheter 1105, and/or other structures in the subject, volume, or region of interest. The returned ultrasound energy waves are sensed by the acoustic receiver 1170, which can sense and receive ultrasound energy in the diminished-frequency range. In some embodiments, the acoustic receiver can also sense and receive ultrasound energy at the fundamental frequency and/or in the harmonic frequency range. For example, the acoustic receiver 1170 can be a broadband acoustic receiver. Alternatively, the acoustic receiver 1170 can be a narrowband acoustic receiver In a specific example, the acoustic receiver 1170 can have a responsivity that peaks at about 5 MHz and can have responsivity down to about 10 kHz.

The acoustic receiver 1170 can be located inline and/or coaxially with the transducer(s) 1112 of the ultrasound device 1110, such that the ultrasound energy produced by the ultrasound device 1110 passes through the acoustic receiver 1170 and/or that the ultrasound energy produced by the ultrasound device 1110 and the returned ultrasonic energy received by the acoustic receiver 1170 are aligned along or parallel to an axis 1175. In this embodiment, the acoustic receiver 1170 is placed on the skin surface of the subject (e.g., via an acoustic coupling medium such as a gel) and the ultrasound device 1110 is placed on the acoustic receiver 1170. Thus, the acoustic receiver 1170 can be located between the skin surface and the transducer(s) 1112. In another embodiment, the broadband acoustic receiver 1170 and the transducer(s) 1112 can be positionally offset from one another in which case the ultrasound energy produced by the ultrasound device 1110 bypasses the broadband acoustic receiver 1170.

The acoustic receiver 1170 is preferably acoustically transparent to the ultrasound energy produced by the ultrasound device 1110. For example, the acoustic receiver 1170 can comprise or consist of polyvinylidene fluoride (PVDF) film.

The output of the acoustic receiver 1170 is electrically coupled to the input of the preamplifier 1120, which amplifies the broadband signals sensed or received by the acoustic receiver 1170. The preamplifier 1120 can be optional in some embodiments. The output of the preamplifier 1120 is electrically coupled to the input of the low-pass filter 1130.

The low-pass filter 1130 is configured to filter out the broadband signals that represent the returned ultrasound energy waves having the fundamental frequency and harmonic-frequencies, resulting in filtered diminished frequencies. The low-pass filter 1130 can have a bandwidth of less than f₀ to remove the fundamental frequency. For example, the low-pass filter 1130 can have an f₀/2 bandwidth. The filter 1130 can be analog or digital, or both, and can be optional in some embodiments. The low-pass filter 1130 can include or can be replaced with a bandpass filter.

The output of the low-pass filter 1130 is electrically coupled to the input of the gain amplifier 1140 to amplify the filtered diminished-frequency signals. The amplifier 1140 can be optional in some embodiments. The output of the gain amplifier 1140 is electrically coupled to the input of the ADC 1150 to produce a digital representation of the filtered diminished-frequency signals. The digital representation data can be acquired by the memory 1160, which can be computer-readable memory with precise triggers to begin writing/storing data in the memory 1160, such as through a field-programmable gate array (FPGA) 1180 or similar/equivalent in electrical communication with the memory 1160. The FPGA 1180 can be configured to produce the trigger signal, analyze, and/or store the digital diminished-frequency representations in memory. The memory 1160 can be used to acquire multiple large sequential signals to look at the time evolution of data. The memory 1160 can be optional in some embodiments. The memory 1160 can include segmented memory and/or unsegmented memory. In general, the signal takes more than 1 insonation-emission cycle to form and reach its maximum signal-to-background (S/B) ratio.

A computer 1190 can be electrically coupled to, operatively coupled to, and/or can include the memory 1160. The computer 1190 can automatically produce a graphical display of the digital data stored in memory 1160, such as on a display screen 1192. Additionally or alternatively, the computer 1190 can monitor and/or analyze the data stored in memory 1160 and determine or detect when the digital diminished-frequency representations data includes a diminished-frequency spectral signature.

The computer 1190 includes a microprocessor 1194 and non-volatile computer-readable memory 1196. The microprocessor 1194 is in electrical communication with the memory 1196 to retrieve and execute computer-readable instructions, such as software, stored thereon, to perform one or more tasks as described herein.

The computer 1190 can produce an output control signal that indicates that a diminished-frequency spectral signature is detected. The output control signal can produce an audible feedback signal, a visual feedback signal (e.g., on display screen 1192), a haptic feedback signal, and/or another feedback signal to alert a user of the system 1100. The computer 1190 can be configured to automatically determine a spatial location of the target 1101 (e.g., biomineralization) based, at least in part, on the diminished-frequency spectral signature. For example, the spatial location of the target 1101 can be determined using the spatial position and orientation of the acoustic receiver 1170 and/or of the transducer(s) 1112 at the time that the diminished-frequency spectral signature is detected.

The computer 1190 can optionally be in electrical communication with the ultrasound device 1110, for example to send control signals to the ultrasound device 1110 to start and/or stop the ultrasound device 1110 from producing ultrasound energy (e.g., for biomineralization/target 1101 detection or treatment), to control the ultrasound device 1110 while the diminished-frequency spectral signature is detected and/or to control the ultrasound device 1110 during therapeutic treatment. In one example, the computer 1190 can automatically cause the ultrasound device 1110 to begin therapeutic treatment of the biomineralization/target 1101 after detecting the diminished-frequency spectral signature.

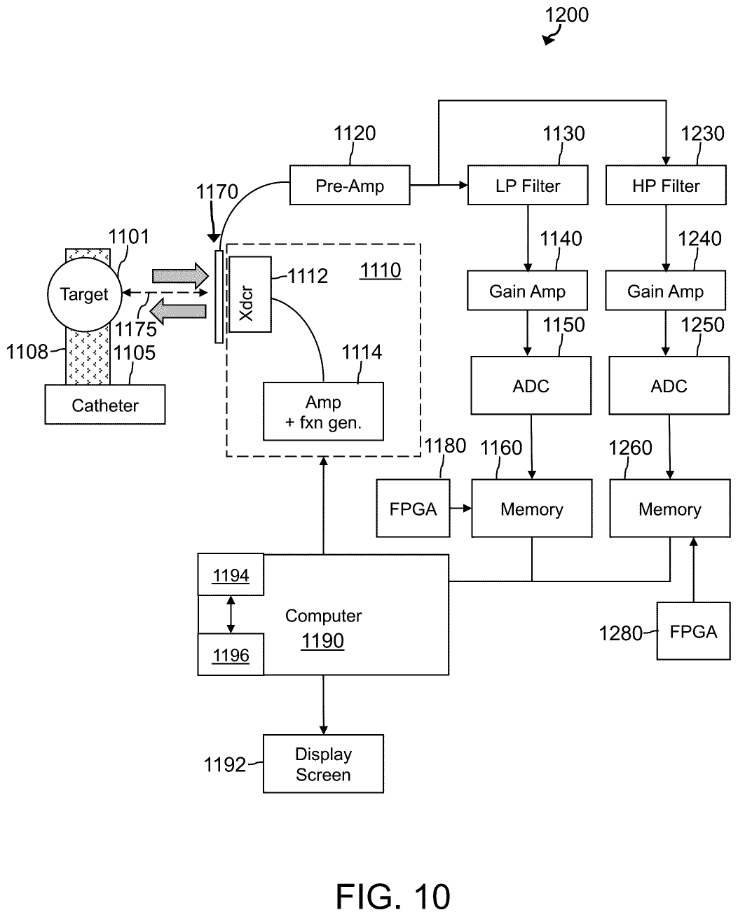

FIG. 10 is a block diagram of a system 1200 for localizing a biomineralization in a volume according to an embodiment. System 1200 is configured to simultaneously detect multiple frequency ranges. System 1200 is the same as system 1100 except that system 1200 includes a high-pass filter 1230, a gain amplifier 1240, an ADC 1250, and memory 1260. The input of the high-pass filter 1230 is electrically coupled to the output of the preamplifier 1120. Thus, the output of the preamplifier 1120 is electrically coupled to both the input of the low-pass filter 1130 and to the input of the high-pass filter 1230.

The high-pass filter 1230 is configured to filter out the lower-band signals that represent the returned ultrasound energy having diminished frequencies, resulting in filtered fundamental and harmonic frequencies. The output of the high-pass filter 1230 is electrically coupled to the input of the gain amplifier 1240 to amplify the filtered fundamental and harmonic frequencies.

The output of the gain amplifier 1240 is electrically coupled to the input of the ADC 1250 to produce a digital representation of the filtered fundamental and harmonic frequency signals. The digital representation data can be acquired by the memory 1260, which can be computer-readable memory with precise triggers to write data at specific time points, such as through a FPGA 1280 or similar/equivalent. Memory 1260 can be the same as memory 1160. The memory 1260 can be used to acquire multiple large sequential signals to look at the time evolution of data. The computer 1190 can be electrically coupled to the memory 1260. The filtered fundamental and harmonic frequencies can be automatically analyzed and/or displayed by the computer 1190 such as for diagnostic imaging. For example, the filtered fundamental and harmonic frequencies can correspond to one or more diagnostic images that can be automatically produced by the computer 1190 and displayed on the display screen 1192. Memory 1160 and/or 1260 can be optional in some embodiments.

The amplifier 1140 and/or 1240 can be optional in some embodiments. The low-pass filter 1130 and/or the high-pass filter 1230 can be optional in some embodiments.

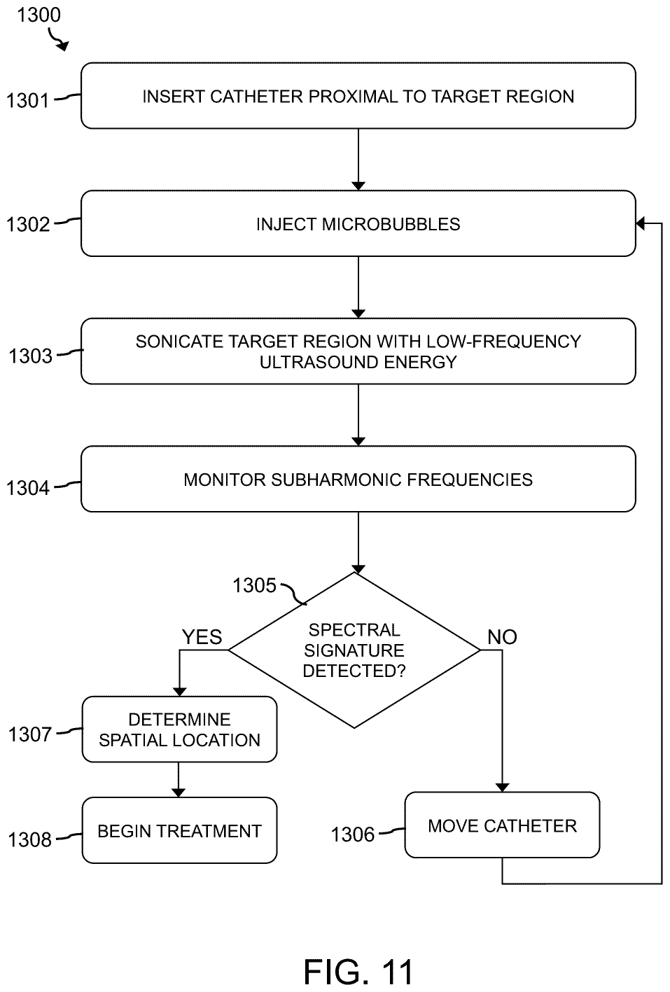

FIG. 11 is a flow chart of a method 1300 for localizing a biomineralization, such as a urinary stone, in a volume according to an embodiment. Method 1300 can be performed using system 1100 and/or system 1200. In step 1301, a catheter is placed or inserted near the region of interest or volume, such as the ureter, of the patient or subject. In step 1302, an ensemble of microbubbles is injected from the catheter, for example using a pump or syringe, such as pump/syringe 150. In step 1303, the target region is sonicated with low-frequency ultrasound energy using an ultrasound device. For example, the ultrasound energy can have a center or fundamental frequency of less than about 1 MHz, such as about 250 kHz to about 750 kHz. In a specific example, the ultrasound energy can have a center or fundamental frequency of about 500 kHz. Steps 1302 and 1303 can occur simultaneously.

In step 1304, diminished frequencies are monitored for the indication of clustered microbubbles, such as microbubble clouds, which may form on and/or around the undesired biomineralization. The diminished frequencies can be monitored using a low-pass filter and associated electronics, for example in system 1100 and/or system 1200, including a processor and/or computer (e.g., computer 1190). Additionally or alternatively, the fast Fourier transform (FFT) of the received ultrasound energy can be taken with respect to the diminished frequencies of interest (e.g., in the range above 0 Hz and below f₀/2). The FFT can be performed by the processor and/or computer (e.g., computer 1190 and/or FPGA(s) 1180, 1280). The FFT can be taken of a digital representation of a frequency output of a receiver that receives the returned ultrasound energy waves. Additionally or alternatively, the diminished frequencies can be monitored by analog and/or digital filtering of the returned ultrasonic energy waves.

In step 1305, it is automatically determined whether a diminished-frequency spectral signature is detected (e.g., using computer 1190). The diminished-frequency spectral signature can indicate whether the microbubbles have clustered on and/or around the undesired biomineralization, such as in a microbubble cloud. An example of a diminished-frequency spectral signature is a decrease or dip in the diminished-frequency response along an axis (e.g., as illustrated in FIG. 2). Another example of a diminished-frequency spectral signature an increased pressure response at the biomineralization surface compared to other locations and/or a different time dependence on versus off the biomineralization surface. Additional examples of diminished-frequency spectral signatures are described herein.

When a diminished-frequency spectral signature is not detected (i.e., step 1305=no), the catheter is moved in step 1306 so as to inject the microbubbles at a different location, which occurs when steps 1302-1305 are repeated. The ultrasound device can also be moved in step 1306. When a diminished-frequency spectral signature is detected (i.e., step 1305=yes), the spatial location of the biomineralization is determined in step 1307. The spatial location can be determined using a processor and/or computer (e.g., computer 1190 ) and is based, at least in part, on the diminished-frequency spectral signature. After the spatial location of the biomineralization is determined, therapeutic treatment can begin in optional step 1308.

This method also can be used to localize and/or track a catheter, an endoscope, a medical device, and/or a placement device within a working channel by using the diminished-frequency spectral signature of microbubbles or fluid containing cavitation nuclei. Said fluid can be contained or dispensed via the catheter or the working channel of the endoscope of the medical device or of the placement device. Localization of a catheter, endoscope working channel, or medical device working channel, and/or placement device working channel can aid in the alignment during treatment procedures, e.g., ureteroscopic lithotripsy procedures to aid in measurement of distances and placement guidance, or placement of other intra-corporeal devices, for example stents.

This diminished-frequency spectral signature method can be further used to localize biomineralization fragments using microbubbles which accumulate on the fragments, e.g., via a targeting moiety or other methods and indicate their presence and/or location.

This method can be further used to measure the diameter of the channel using the lifetime of the diminished-frequency spectral signature signal. Larger channels produce a longer-lived signal compared to smaller channels. Measurement of this lifetime can then be related to intra-operative conditions, such as a stricture of the ureter, hydroureter, or dilation/constriction of other vessels of the human body.

The detection methods and systems of this patent can be combined with other detection methods and/or detection systems to produce a combined method and/or system to improve sensitivity, responsivity, and/or localization. This combined method and/or system functionally relates signals to correctly identify microbubbles which in turn can be related to larger scale structures.

This combination may include combining the returned-frequency signal with other signals such as the fundamental (f0) and/or higher harmonics (2f0) and/or broadband emission (e.g. in between 1 MHz and 1.5 MHz).

The detection methods and/or systems of this combination can be combined with other detection methods and/or systems where the other detection methods and/or systems is/are an acoustic detection method/system, e.g. ultrasound, electromagnetic detection, e.g. x-ray.

The detection methods and/or systems of this combination can improve image analysis and/or pattern recognition.

In some embodiments, spontaneous cavitation of the microbubbles is used. The cavitation nuclei used in this system and/or method are not performed but are generated through the application of ultrasound.

In some embodiments, a method and/or system for treatment and alignment is/are disclosed. The method and/or device include the disclosed diminished-frequency detection method and/or system, which can be combined with one or more of the following: (a) a method and/or system for aligning a transducer to a target; (b) a method and/or system for positioning a catheter relative to an obstructing stone; and/or (c) a method and/or system for monitoring the positioning and alignment during treatment.

In an aspect, this disclosure provides a method for treating a medical condition involving an abnormal or obstructive mass. In some embodiments, the medical condition involves kidney stones, urinary stones, biliary stones, blood clots, fibroids, cancerous tumors, and/or atheromatous plaques. In some embodiments, the subject has urolithiasis.

In some embodiments, the method comprises: administering to a subject with the medical condition an effective amount of the microbubbles, such as an ensemble of microbubbles, so as to bring the microbubbles into contact with the abnormal or obstructive mass (e.g., urinary stone, kidney stone, biliary stones, blood clots, fibroids, cancerous tumors, and/or atheromatous plaques); and directionally applying an energy, at a frequency that excites the fluid within the microbubbles, to the abnormal or obstructive mass within the subject.

In some embodiments, the microbubbles are administered into the ureter of the subject through a urinary catheter. In some embodiments, the applied energy is in the form of electromagnetic, acoustic (e.g., ultrasound), microwave, photonic, laser, or other forms.

The invention should not be considered limited to the particular embodiments described above. Various modifications, equivalent processes, as well as numerous structures to which the invention may be applicable, will be readily apparent to those skilled in the art to which the invention is directed upon review of this disclosure. The above-described embodiments may be implemented in numerous ways. One or more aspects and embodiments involving the performance of processes or methods may utilize program instructions executable by a device (e.g., a computer, a processor, or other device) to perform, or control performance of, the processes or methods.

In this respect, various inventive concepts may be embodied as a non-transitory computer readable storage medium (or multiple non-transitory computer readable storage media) (e.g., a computer memory of any suitable type including transitory or non-transitory digital storage units, circuit configurations in Field Programmable Gate Arrays or other semiconductor devices, or other tangible computer storage medium) encoded with one or more programs that, when executed on one or more computers or other processors, perform methods that implement one or more of the various embodiments described above. When implemented in software (e.g., as an app), the software code may be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more communication devices, which may be used to interconnect the computer to one or more other devices and/or systems, such as, for example, one or more networks in any suitable form, including a local area network or a wide area network, such as an enterprise network, and intelligent network (IN) or the Internet. Such networks may be based on any suitable technology and may operate according to any suitable protocol and may include wireless networks or wired networks.

Also, a computer may have one or more input devices and/or one or more output devices. These devices can be used, among other things, to present a user interface. Examples of output devices that may be used to provide a user interface include printers or display screens for visual presentation of output and speakers or other sound generating devices for audible presentation of output. Examples of input devices that may be used for a user interface include keyboards, and pointing devices, such as mice, touch pads, and digitizing tablets. As another example, a computer may receive input information through speech recognition or in other audible formats.

The non-transitory computer readable medium or media may be transportable, such that the program or programs stored thereon may be loaded onto one or more different computers or other processors to implement various one or more of the aspects described above. In some embodiments, computer readable media may be non-transitory media.

The terms “program,” “app,” and “software” are used herein in a generic sense to refer to any type of computer code or set of computer-executable instructions that may be employed to program a computer or other processor to implement various aspects as described above. Additionally, it should be appreciated that, according to one aspect, one or more computer programs that when executed perform methods of this application need not reside on a single computer or processor, but may be distributed in a modular fashion among a number of different computers or processors to implement various aspects of this application.

Computer-executable instructions may be in many forms, such as program modules, executed by one or more computers or other devices. Generally, program modules include routines, programs, objects, components, data structures, etc. that performs particular tasks or implement particular abstract data types. The functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Thus, the disclosure and claims include new and novel improvements to existing methods and technologies, which were not previously known nor implemented to achieve the useful results described above. Users of the method and system will reap tangible benefits from the functions now made possible on account of the specific modifications described herein causing the effects in the system and its outputs to its users. It is expected that significantly improved operations can be achieved upon implementation of the claimed invention, using the technical components recited herein.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

What is claimed is:

1. A method for localizing a biomineralization in a volume, comprising:
producing, by an ultrasonic transducer, pulses of produced ultrasonic energy waves having a fundamental frequency;
injecting an ensemble of microbubbles proximal to the biomineralization;
receiving, by an acoustic receiver, returned ultrasonic energy waves to detect a broadband signal output;
processing the broadband signal output to isolate diminished frequencies of the broadband signal output, the diminished frequencies lower than 50% of the fundamental frequency;
monitoring the diminished frequencies, with a processor, for a diminished-frequency spectral signature that corresponds with a location of the biomineralization; and
determining a spatial location of the biomineralization, with the processor, based on the diminished-frequency spectral signature.

2. The method of claim 1, wherein the diminished frequencies are less than or equal to 25% of the fundamental frequency.

3. The method of claim 1, wherein the fundamental frequency is greater than or equal to about 250 kHz and less than about 1 MHz.

4. The method of claim 3, wherein the fundamental frequency is less than or equal to about 750 kHz.

5. The method of claim 3, wherein the fundamental frequency is about 500 kHz.

6. The method of claim 5, wherein the diminished frequencies are greater than or equal to about 20 kHz and less than or equal to about 120 kHz.

7. The method of claim 1, wherein the diminished-frequency spectral signature includes an increased acoustic pressure response of the diminished frequencies when the produced ultrasonic energy waves are aligned with the biomineralization compared to when the produced ultrasonic energy waves are offset from the biomineralization.

8. The method of claim 7, wherein the increased acoustic pressure response of the diminished frequencies occurs when the microbubbles accumulate on a surface of the bio mineralization.

9. The method of claim 8, wherein the increased acoustic pressure response of the diminished frequencies occurs when the microbubbles form a microbubble cloud and/or a microbubble cluster.

10. The method of claim 1, wherein the diminished-frequency spectral signature includes an integrated signal of an acoustic pressure of the diminished frequencies.

11. The method of claim 1, further comprising moving the ultrasound transducer axially with respect to the volume while the ultrasound transducer produces the pulses of produced ultrasonic energy waves,
wherein the diminished-frequency spectral signature includes:
a first increase in a diminished-frequency response over a first time period, the first increase in the diminished-frequency response compared to a background response signal,
a decrease in the diminished-frequency response over a second time period compared to the diminished-frequency response over the first time period, and
a second increase in the diminished-frequency response over a third time period, the second increase in the diminished frequency response compared to the background response signal,
wherein:
the second time period immediately follows the first time period, and
the third time period immediately follows the second time period.

12. The method of claim 11, wherein the decrease in the diminished-frequency response over the second time period corresponds to the spatial location of the bio mineralization.

13. The method of claim 12, wherein the first and second increases in the diminished-frequency response correspond to a spatial location of microbubbles and/or a microbubble cloud.

14. The method of claim 1, wherein the diminished frequencies are greater than or equal to about 4% of the fundamental frequency.

15. The method of claim 1, further comprising amplifying the diminished frequencies of the broadband signal output.

16. The method of claim 1, wherein the processing step includes filtering, in a low-pass filter or a bandpass filter, the broadband signal output to isolate the diminished frequencies.

17. The method of claim 1, wherein the processing step includes performing, with the processor, a fast Fourier transform of the broadband signal output with respect to the diminished frequencies.

18. A system for localizing a biomineralization in a volume, comprising:
an ultrasound device that produces pulses of produced ultrasonic energy waves having a fundamental frequency;
a receiver that receives returned ultrasonic energy waves and produces signals that represent the returned ultrasonic energy waves;
a low-pass filter configured to receive the signal output of the receiver and to isolate diminished frequencies of the signal output, the diminished frequencies less than 50% of the fundamental frequency;
an analog-to-digital converter configured to convert the diminished frequencies to digital diminished-frequency representations;
a non-volatile computer-readable memory that stores the digital diminished-frequency representations;
a catheter configured to inject an ensemble of microbubbles proximal to the biomineralization; and
a computer having a microprocessor and non-volatile computer-readable memory, the computer operatively coupled to the computer-readable memory, the non-volatile computer-readable memory storing computer-readable instructions that, when executed by the microprocessor, cause the microprocessor to automatically:
determine whether the digital diminished-frequency representations include a diminished-frequency spectral signature that corresponds with a spatial location of a biomineralization in a volume, and
produce an output control signal that indicates that the diminished-frequency spectral signature is detected.

19. The system of claim 18, further comprising:
a preamplifier having an input electrically coupled to an output of the receiver, the preamplifier having an output electrically coupled to an input of the low-pass filter; and
a gain amplifier having an input electrically coupled to an output of the low-pass filter, the gain amplifier having an output electrically coupled to an input of the analog-to-digital converter.

20. The system of claim 18, further comprising a field-programmable gate array (FPGA) in electrical communication with the memory, the FPGA configured to produce a trigger signal to store the digital diminished-frequency representations in the memory.

21. The system of claim 18, wherein:
the analog-to-digital converter is a first analog-to-digital converter,
the non-volatile computer-readable memory is a first non-volatile computer-readable memory, and
the system further comprises:
a high-pass filter configured to receive the signal output of the receiver and to isolate the fundamental and harmonic frequencies of the signal output;
a second analog-to-digital converter configured to convert the fundamental and harmonic frequencies to digital fundamental and harmonic representations; and
a second non-volatile computer-readable memory that stores the digital fundamental and harmonic representations,
wherein the computer-readable instructions, when executed by the microprocessor, further cause the microprocessor to produce a diagnostic image on a display screen in electrical communication with the computer, the diagnostic image corresponding to the digital fundamental and harmonic representations.

22. The system of claim 18, wherein the ultrasound device and the receiver are coaxially aligned such that the produced ultrasonic energy waves from the ultrasound device pass through the receiver before passing into the volume.

23. The system of claim 18, wherein the diminished frequencies are greater than or equal to about 4% of the fundamental frequency.

24. A system for localizing a biomineralization in a volume, comprising:
an ultrasound device that produces pulses of produced ultrasonic energy waves having a fundamental frequency;
a receiver that receives returned ultrasonic energy waves and produces signals that represent the returned ultrasonic energy waves;
an analog-to-digital converter configured to convert the signals to digital frequency representations;
a non-volatile computer-readable memory that stores the digital frequency representations;
a catheter configured to inject an ensemble of microbubbles proximal to the biomineralization; and
a computer having a microprocessor and non-volatile computer-readable memory, the computer operatively coupled to the segmented computer-readable memory, the non-volatile computer-readable memory storing computer-readable instructions that, when executed by the microprocessor, cause the microprocessor to automatically:
perform a fast Fourier transform of the digital frequency representations with respect to diminished frequencies that are less than 50% of the fundamental frequency to isolate the diminished frequencies,
determine whether a diminished-frequency signal corresponding to the diminished frequencies includes a diminished-frequency spectral signature that corresponds with a spatial location of a biomineralization in a volume, and
produce an output control signal that indicates that the diminished-frequency spectral signature is detected.
